(19) United States
(12) Patent Application Publication
(10) Pub. No.: US 2009/0118833 A1
(43) Pub. Date: May 7, 2009

(54) IN-SITU CURABLE INTERSPINOUS PROCESS SPACER

(75) Inventors: Robert G. Hudgins, Monticello, MN (US); Hugh D. Hestad, Edina, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/934,984

(22) Filed: Nov. 5, 2007

(51) Int. Cl. A61F 2/44; A61M 29/00

(52) U.S. Cl. 623/17.16; 606/192; 623/17.12

(57) ABSTRACT

The present invention provides an expandable member useful in treating spinal stenosis. The expandable member may be introduced into the patient between adjacent spinous processes in an unexpanded configuration using minimally invasive techniques and expanded with a flowable material. The expanded member will function as an interspinous process spacer by acting as a spacing device to maintain separation between adjacent vertebrae.

### FIELD OF THE INVENTION

The present invention relates generally to devices for treating spinal stenosis, and more particularly to interspinous process spacers that can be implanted in a minimally invasive manner to treat spinal stenosis.

### BACKGROUND OF THE INVENTION

A large majority of the population will experience back pain at some point in their lives that results from a spinal condition. The pain may range from general discomfort to disabling pain that immobilizes the individual. One type of adverse spinal condition is spinal stenosis which occurs when the spinal canal or nerve root canals become too narrow and reduces the space for the passage of blood vessels and nerves.

Lumbar spinal stenosis (“LSS”, and sometimes called sciatica) is a condition of the spine characterized by a narrowing of the lumbar spinal canal. With lumbar spinal stenosis, the spinal canal narrows and pinches the spinal cord and nerves, causing pain in the back and legs. It is estimated that approximately 5 in 10,000 people develop LSS each year. For patients who seek the aid of a physician specialist for back pain, approximately 12-15% are diagnosed as having LSS.

Several causes of spinal stenosis have been identified, including aging, heredity, arthritis, and changes in blood flow to the lower spine. Aging is believed to be the most common cause, because as a person ages the ligaments connecting the bones of the spine can thicken and spurs may develop on the bones and into the spinal canal. The cushioning discs between the vertebrae also frequently deteriorate, and the facet joints may begin to break down. Over time, loss of disk height in the lumbar regions can result in a degenerative cascade with deterioration of all components of a motion segment resulting in segment instability and ultimately in spinal stenosis. During the process of deterioration, disks can become herniated and/or become internally torn and chronically painful. When symptoms seem to emanate from both anterior (disk) and posterior (facets and foramen) structures, patients cannot tolerate positions of extension or flexion. Heredity is believed to play a role in some cases because it may cause some people to have a smaller than average spinal canal, typically leading to LSS symptoms even at a relatively young age.

The most common symptoms of spinal stenosis are pain and difficulty when walking, although numbness, tingling, hot or cold feelings in the legs, and weakness or tiredness may also be experienced. In extreme cases, spinal stenosis can cause cauda equina syndrome, a syndrome characterized by neuromuscular dysfunction that may result in permanent nerve damage.

Common treatments for LSS include physical therapy (including changes in posture), medication, and occasionally surgery. Changes in posture and physical therapy may be effective in flexing the spine to enlarge the space available to the spinal cord and nerves—thus relieving pressure on pinched nerves. Medications such as NSAIDS and other anti-inflammatory medications are often used to alleviate pain, although they are not typically effective at addressing the cause of the pain. Surgical treatments are more aggressive than medication or physical therapy, but in appropriate cases surgery may be the best way to achieve a lessening of the symptoms associated with LSS.

The most common surgery for treating LSS is decompressive laminectomy, in which the lamina of one or more vertebrae is removed to create more space for the nerves. The intervertebral disc may also be removed, and the vertebrae may be fused to strengthen unstable segments. The success rate of decompressive laminectomy has been reported to be in excess of 65%, with a significant reduction in LSS symptoms being achieved in many cases.

More recently, a second surgical technique has been developed in which the vertebrae are distracted and an interspinous process spacer is implanted to maintain the desired separation between the segments. This technique is somewhat less invasive than decompressive laminectomy, but may provide significant benefits to patients experiencing LSS symptoms.

As with other surgeries, one consideration when performing surgery to implant an interspinous process spacer is the size of the incision that is required to allow introduction of the device. Medical treatments that can be performed in a minimally invasive manner are greatly sought after by the medical community and patients alike. The term “minimally invasive” herein shall be understood as being accomplished by providing a technique less invasive than an open procedure to gain access to the application point. In some procedures, minimally invasive techniques are advantageous because there may be no need to resect tissue so that they can be performed with the use of a local anesthesia, have a shorter recovery period, result in little to no blood loss, and greatly decrease the chances of significant complications. Additionally, many minimally invasive techniques may not require the use of general anesthesia, thereby avoiding the associated risks. Moreover, minimally invasive techniques are usually less expensive for the patient.

Therefore, minimally invasive techniques are generally preferred, but several interspinous process spacers previously known in the art do not work well with minimally invasive surgical techniques. The implantation profile presented by known spacers precludes introduction through a very small incision. A need therefore exists for an interspinous process spacer that can be implanted using minimally invasive surgical techniques. Moreover, it would be most desirable to be able to perform this procedure using arthroscopic techniques.

In view of the many advantages of arthroscopic procedures, it would be highly advantageous to have an interspinous process spacer and an associated procedure amenable to arthroscopic techniques. The present invention addresses that need.

### SUMMARY OF THE INVENTION

The present invention addresses these and other problems associated with the prior art by providing a customized interspinous process spacer and associated method to insert it into a medical patient with a minimally invasive procedure. The spacer is to act as a spacing device for the spinous processes of two adjacent vertebrae. The interspinous process spacer is used to distract the vertebrae and relieve pressure on the posterior wall of the intervertebral disc. Furthermore, the spacer is expected to relieve pain associated with the spinal canal and/or neural foramen stenosis as well as potentially relieving pain associated with degenerative facet joints. The interspinous process spacer of the present invention will allow controlled flexion and limited extension at the implanted level.

A first aspect of the present invention is a method for implanting a customized interspinous process spacer for maintaining separation between adjacent superior and inferior spinous processes of two adjacent vertebrae. The method comprises introducing an expandable member between the adjacent superior and inferior spinous processes. The expandable member is introduced percutaneously or arthroscopically while the expandable member is in an unexpanded configuration. The expandable member is expanded to a geometry, corresponding to a desired space to be occupied between the adjacent superior and inferior spinous processes, by introducing a measured amount of a flowable material via a catheter to fill the expandable member to the geometry. Time is provided to allow the delivered flowable material to cure and after sufficient curing, the catheter body is severed from the expandable member portion containing the cured material.

Yet another aspect of the present invention is a method for sizing an interspinous process spacer. The sizing method comprises introducing an expandable sizing member between the adjacent superior and inferior spinous processes and introducing a fluid into the expandable sizing member in an amount corresponding to a desired space to be occupied between the adjacent superior and inferior spinous processes. The amount of introduced fluid is used to determine an amount of flowable material necessary to fill the expandable member to the desired space.

### BRIEF DESCRIPTION OF THE DRAWINGS

A more complete appreciation of the invention and many of the attendant advantages thereof will become readily apparent with reference to the accompanying drawings. These drawings, which are incorporated in and constitute part of this specification, illustrate embodiments of the invention and, together with a general description of the invention given above, and the detailed description given below, serve to explain the principles of the invention.

FIG. 1A is a rear elevational view of an interspinous process spacer in the form of an expandable member according to one aspect of the present invention, wherein the expandable member is in an unexpanded configuration and positioned between adjacent superior and inferior spinous processes of two adjacent vertebrae;

FIG. 1B is a view similar to FIG. 1A, wherein the expandable member is in its expanded configuration and positioned between adjacent superior and inferior spinous processes of two adjacent vertebrae;

FIG. 2A is a side elevational view of the expandable member shown in FIG. 1A;

FIG. 2B is a side elevational view of the expandable member shown in FIG. 1B;

FIG. 3A is a cross-sectional view taken along lines 3A-3A of FIG. 2A;

FIG. 3B is a cross-sectional view taken along lines 3B-3B of FIG. 2B;

FIG. 4 is a rear elevational view of an interspinous process spacer according to another aspect of the present invention, wherein the spacer is fixed between adjacent superior and inferior spinous processes of two adjacent vertebrae using bone darts and fixation tabs;

FIG. 5A is a side elevational view of an interspinous process spacer according to yet another aspect of the present invention, wherein the spacer is generally H-shaped and positioned between adjacent superior and inferior spinous processes of two adjacent vertebrae.

FIG. 5B is a cross-sectional view taken along lines 5B-5B of FIG. 5A;

FIG. 6 is a rear elevational view of an interspinous process spacer according to another aspect of the present invention, wherein the spacer is fixed between adjacent superior and inferior spinous processes of two adjacent vertebrae using a fiber tied to the superior and inferior spinous processes of two adjacent vertebrae;

FIG. 7A is a side elevational view of an interspinous process spacer according to another aspect of the present invention, wherein the spacer is generally cylindrical in shape and positioned between adjacent superior and inferior spinous processes of two adjacent vertebrae;

FIG. 7B shows is a cross-sectional view taken along lines 7B-7B of FIG. 7A; and

FIG. 8 is a rear elevational view of an interspinous process spacer according to another aspect of the present invention, wherein the spacer is generally cylindrical in shape and positioned between adjacent superior and inferior spinous processes of two adjacent vertebrae.

### DETAILED DESCRIPTION

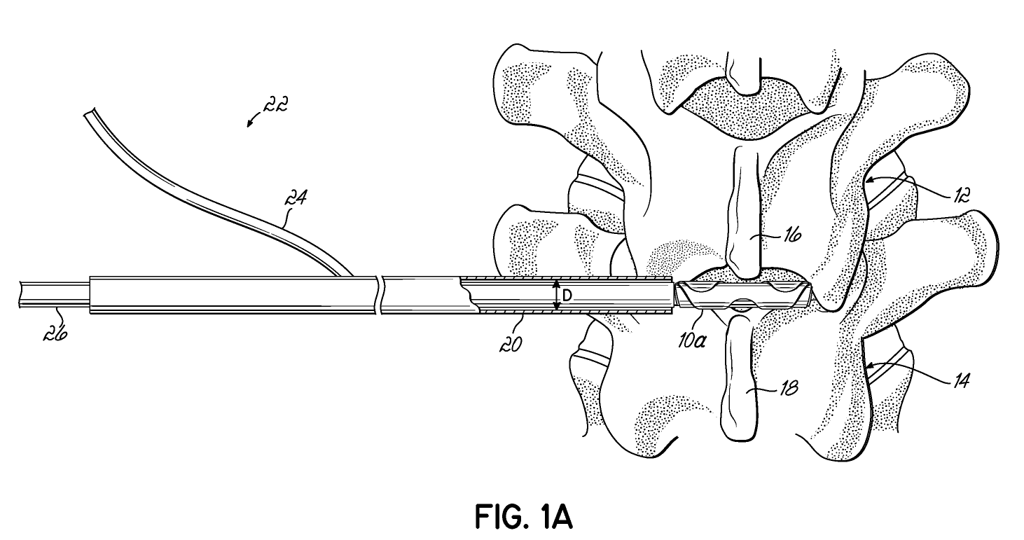

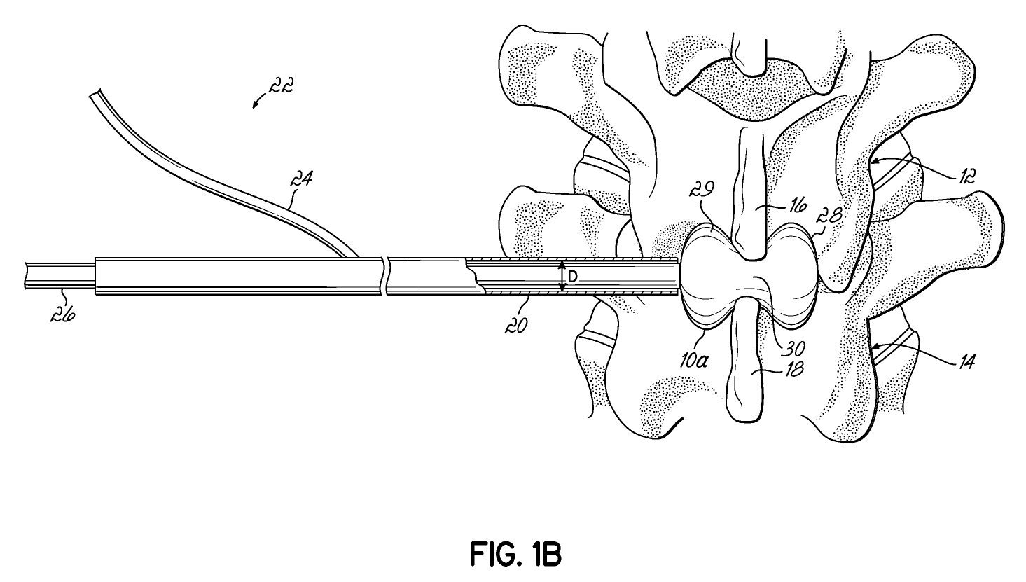

With reference to the Figures, wherein like numbers denote like parts throughout the several views, exemplary interspinous process spacers 10a-c are shown in accordance with the principles of the present invention for maintaining a desired spacing between the spinous processes of adjacent vertebrae 12 and 14. In one embodiment, FIG. 1A illustrates the expandable member 10a in unexpanded form positioned between adjacent superior and inferior spinous processes 16, 18 of the two adjacent vertebrae prior to expansion with a flowable material. The unexpanded, expandable member 10a may be delivered to the desired space through a cannula 20 that defines an access path. In one embodiment, the internal diameter D of cannula 20 is 10 mm or less. In another embodiment the internal diameter D of cannula 20 is 3 mm or less.

The member 10a may be positioned and exposed in the interprocess space by retracting the cannula 20 from the unexpanded, expandable member 10a or extending the unexpanded, expandable member 10a from the cannula 20. Moreover, the cannula 20 may be associated with a venting system 22 having a passageway 24 for maintaining the expandable member 10a in the unexpanded form and a catheter 26 for delivering the flowable material to the expandable member 10a. In another embodiment, FIG. 1B illustrates the expandable member 10a, having a geometry generally in the form of a dumbbell, occupying the interprocess space between the adjacent superior spinous process 16 and the inferior spinous process 18 of two adjacent vertebrae 12, 14 after expanding with flowable material.

As shown in FIG. 1B, expandable member 10a has a generally small medial portion 30 adapted to reside between the adjacent superior spinous process 16 and inferior spinous process 18 to maintain separation therebetween. In addition, expandable member 10a has opposing enlarged lateral portions, including a distal portion 28 and a proximal portion 29, adapted to reside on opposing sides of the adjacent superior and inferior spinous processes 16, 18 to maintain positioning of the member within the interprocess space.

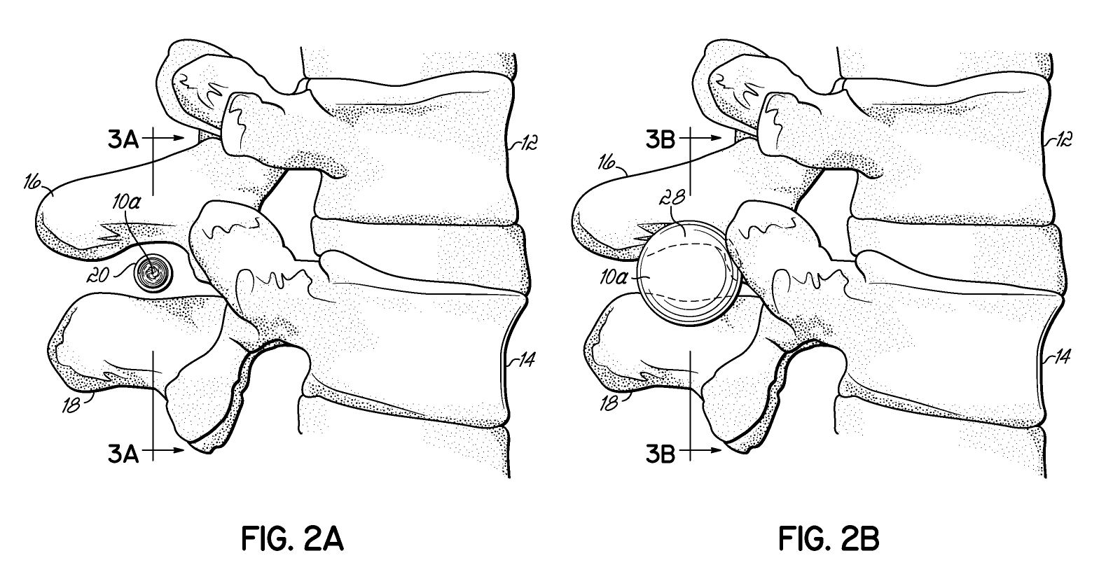

FIG. 2A illustrates a side elevational view of the expandable member 10a, wherein the expandable member 10a is in its unexpanded configuration and positioned between adjacent superior and inferior spinous processes 16, 18 of two adjacent vertebrae 12, 14. FIG. 2B illustrates the distal enlarged lateral portion 28 after inflation of member 11a.

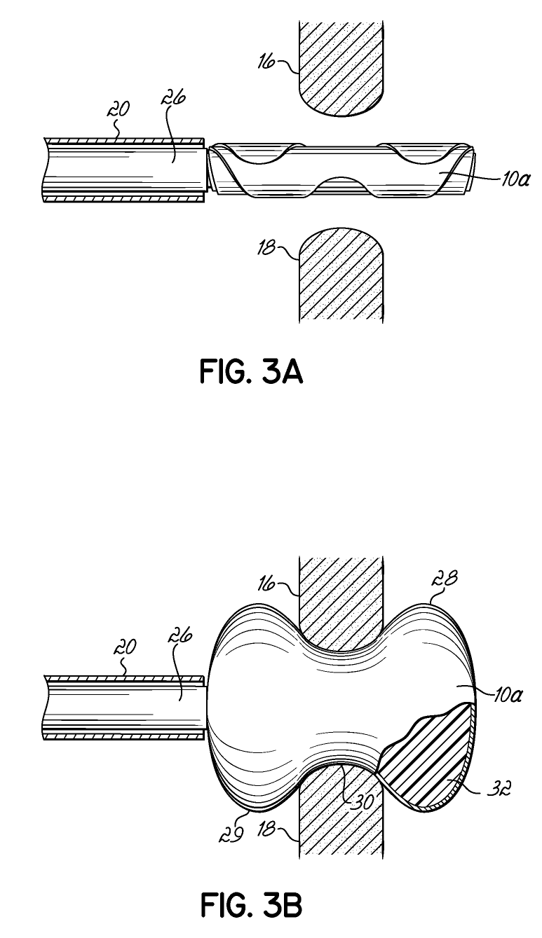

As shown in FIG. 3A, the expandable member 10a, in its unexpanded configuration, is positioned between adjacent superior and inferior spinous processes 16, 18 of two adjacent vertebrae 12, 14. FIG. 3B illustrates the expandable member 10a in its expanded configuration generally in form of a dumbbell, whereby the distal and proximal enlarged lateral portions 28, 29 reside on the side of the adjacent superior and inferior spinous processes 16, 18 to maintain positioning of the member within the desired space. The expandable member 10a is expanded with a flowable material 32.

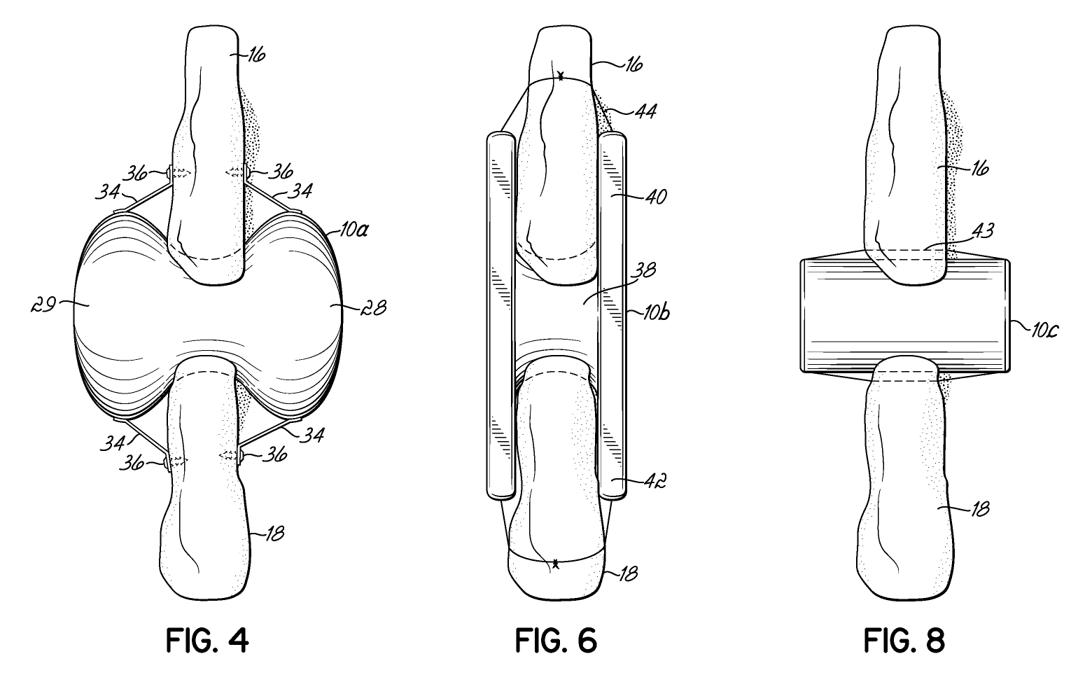

FIGS. 4, 6, and 8 illustrate three exemplary embodiments of the present invention and various means for fixing the interspinous process spacer in the desired position. FIG. 4 illustrates a spacer comprising the dumbbell-shaped expandable member 10a with connecting members 34 utilized for fixation of the spacer in the interprocess space. In this embodiment, connecting members 34 are attached to the expandable member 10a on the superior and inferior surfaces of the distal and proximal enlarged lateral portions 28, 29 and are attached to the superior and inferior spinous process by fasteners 36, such as bone darts.

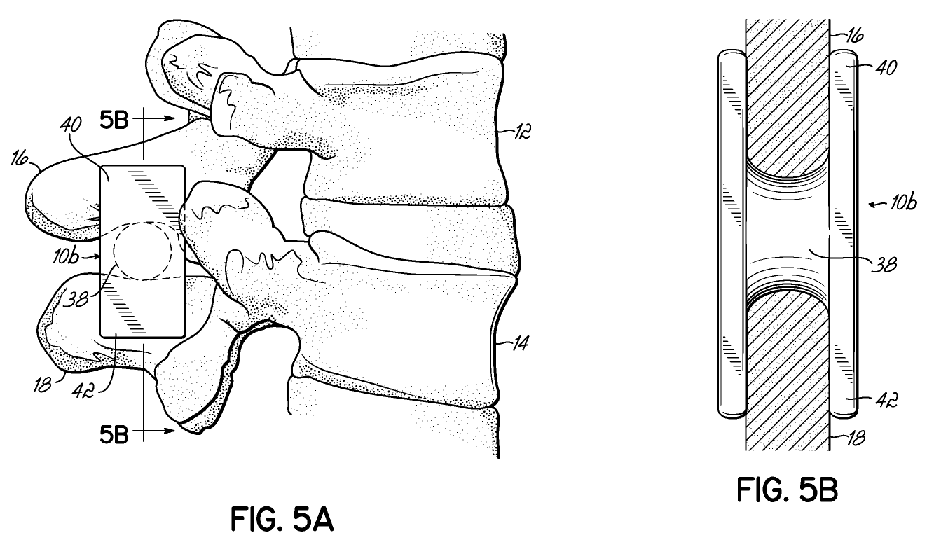

FIGS. 5A, 5B and 6 illustrate an interspinous process spacer comprising an expandable member 10b, having a geometry generally in the form of an H-shape upon expansion. The H-shaped expandable member 10b comprises a generally small medial portion 38 adapted to reside between the adjacent superior and inferior spinous processes 16, 18 to maintain separation therebetween and opposing lateral portions. Each lateral portion includes a superior lateral portion 40 adapted to reside on the lateral side of the superior spinous process 16 and an inferior lateral portion 42 adapted to reside on the lateral side of the inferior spinous process 18. The lateral portions are configured to maintain positioning of the spacer within the interprocess space. As shown in FIG. 6, the fixation of the spacer may be achieved by tying connecting member fibers 44, attached to portions 40 and 42, around the adjacent spinous processes 16 and 18.

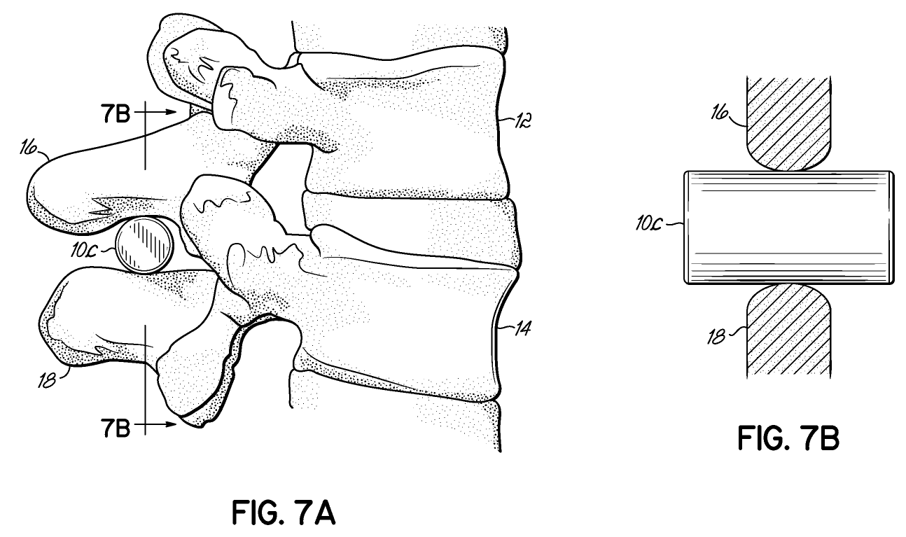

FIGS. 7A, 7B and 8 illustrate a spacer comprising an expandable member 10c, having a geometry generally in the form of a cylinder. As shown in FIG. 8, the spacer may be fixed in the interprocess space by connecting members in the form of sutures 43 that anchor the expandable member 10c to neighboring biological tissue, i.e. sutured to adjacent soft tissue such as the interspinous and supraspinous ligament (not shown.) It will be appreciated that the manner of fixation is not limited to the exemplary embodiments shown in FIGS. 4, 6, and 8. In an alternative embodiment, the expandable member 10a-c may be designed with tissue in-growth capability for long-term fixation, if desired.

In one embodiment, the expandable member 10a-c may be a balloon designed to have a desired geometry upon filling with a flowable material. Moreover, the expandable member 10a-c may be made of non-compliant material to allow generally uniform expansion of the expandable member 10a-c. In another embodiment, the expandable member 10a-c may be made of compliant material that will maintain the desired geometry when expanded. In yet another embodiment, the geometry may be further maintained by casting the expandable member 10a-c with a fiber reinforcing mesh made to the desired geometry of the spacer.

Additionally, the flowable material utilized for expanding the expandable member of the interspinous process spacer may be an in-situ curable material, such as a polymer. In one embodiment, the in-situ curable material may consist of bone cement, polyurethane, silicon, copolymers of silicone and polyurethane, polyolefins, neoprene, nitrile or combinations thereof. The curable material may be chosen based on a surgeon's desired outcome in the patient. For example, a more elastic material may be used to maintain motion in the treatment location. Of course, other suitable fluids are possible as well without departing from the spirit and scope of the present invention.

Alternatively, the expandable member can be filled with, at least in part, a bone growth promoting material that encourages fixation of the expandable member to the spinous processes. In this embodiment, the expandable member can be a mesh material that allows for bone in-growth following alteration of the spinous processes with an instrument such as a rasp. In this embodiment, the bone growth promotion may be incorporated into the in-situ curable polymer providing the benefit of percutaneous delivery and bone in-growth for fixation in a single implant.

The interspinous process spacer of the present invention is suited for implantation using a percutaneous method or another minimally invasive technique versus larger open procedures used for other devices. According to one embodiment, a method for implanting an interspinous process spacer between two adjacent vertebrae comprises the steps of introducing the expandable member 10a-c between the adjacent superior and inferior spinous processes 16, 18 and expanding the member 10a-c to a geometry corresponding to a desired space to be occupied between the adjacent superior and inferior spinous processes 16, 18.

The spacer may be introduced while the expandable member 10a-c is in an unexpanded configuration to facilitate using a minimally invasive surgical procedure, i.e. percutaneously or arthroscopically. The expandable member's orientation and position may be verified radiographically or endoscopically prior to introducing a measured amount of flowable material via the catheter 26 to fill the expandable member 10a-c to a geometry corresponding to a desired space to be occupied between the superior and inferior spinous processes 16, 18. In one embodiment, the flowable material used to fill the expandable member can include a radio-opaque material. Alternatively, radio-opaque markers can be incorporated into the expandable member. After a sufficient amount of time is allowed for the delivered flowable material to cure, the catheter 26 is separated or severed from the expanded member portion 10a-c containing the cured material. Lastly, the interspinous process spacer may be fixed in the interprocess space using at least one connecting member as described in detail above in connection with FIGS. 4, 6 and 8, for example.

According to another aspect of the present invention, a sizing procedure may be used including the steps of introducing an expandable sizing member (not shown) of a geometry corresponding to the desired space between the adjacent superior and inferior spinous processes 16, 18. A fluid is introduced into the sizing member corresponding to a desired space to be occupied between the adjacent superior and inferior processes 16, 18, wherein the amount of fluid is measured and used to determine an amount of flowable material necessary to fill the expandable member 10a-c to the desired geometry in the desired space. The degree of distraction can be verified radiographically prior to unexpanding the sizing member. Finally, the sizing member may be unexpanded by removing the fluid and then it may be withdrawn from the interspinous process space. It may be desirable to use minimally invasive techniques to perform dissection or dilation of tissue to create a desired space around the sizing member to accommodate the resultant interspinous process spacer.

The interspinous process spacer may be sized prior to placement using an expandable sizing member by first making a small skin incision slightly lateral to the mid-point between the desired spinous processes. A guide probe may be inserted through the muscles and the interspinous ligament to the opposite side of the spinous process. The working cannula 20 is then placed, and its position may be verified radiographically. If a dumbbell or cylindrically-shaped expandable member 10a, 10c is used, the catheter 26 may simply be placed through the working cannula 20 to the distal side of the spinous process. The working cannula 20 may be withdrawn slightly toward the proximal side of the spinous process to expose the expandable member 10a, 10c.

After the sizing procedure, the expandable member 10a-c may be inserted into the space and filled with the appropriate amount of flowable material determined from the sizing procedure. The elasticity of the spacer, combined with the rigidity or lack of rigidity of its fixation, will control the degree of flexion achieved. The stiffness of the interspinous process spacer will limit the extension of the spine because the device will be placed in compression.

In other embodiments, a different technique may be necessary, such as to accommodate an H-shaped expandable member 10b with a tie fixation method. The working cannula 20 may be placed as described in detail above. Next, the superior portion of the superior spinous process 16 is located. A small skin incision is made and blunt dissection instruments are passed between the process and the traverseospinalis muscles to create a pocket for the lateral portions of the H-shaped member 10b. The pocket must extend from the superior margin of the process to the cannula 20 so that the tissue can accept the superior lateral portion of the H-shaped member 10b. Pockets must be created on both sides of the superior spinous process 16, and the procedure must be repeated to create pockets around the inferior process 18. The expandable sizing member is placed with the proper orientation and the sizing, dilating, and distraction performed. The expandable member 10b is then placed into the interprocess space and the member filled with the flowable material. The opposing superior and inferior lateral portions of the expandable member 10b fill the pockets around the spinous processes. The spacer may be fixed in place by placing a probe through the pocket and retrieving the fiber tie attached to each lateral portion of the spacer. The two ties on the superior lateral portions may be tied together around the superior process 16 and the procedure repeated for securing the spacer to the inferior process 18.

Essentially the same process may be used if a tie fixation method is used with the dumbbell or cylindrically-shaped expandable members 10a, 10c, but the blunt dissection would be less extensive for simply positioning the ties to the outer margin of the process to facilitate tying the interspinous process spacer in place. Alternatively, a very small incision may be made near midline and blunt dissection may be performed to place the expandable member 10a, 10c. Fixation methods could still be performed in a similar manner as described above.

While the present invention has been illustrated by a description of various embodiments and while these embodiments have been described in considerable detail, it is not the intention of the applicants to restrict or in any way limit the scope of the appended claims to such detail. The describe embodiments are simply intended to clarify the technical idea of the present invention. As such, the technical scope of the present invention should not be construed solely on the basis of the specific embodiments described above. Additional advantages and modifications will readily appear to those skilled in the art. The invention in its broader aspects is therefore not limited to the specific details, representative aspects and methods, and illustrative examples shown and described. Accordingly, departures may be made from such details without departing from the spirit or scope of applicant's general inventive concept.

Having described the invention, we claim:

1. A method for implanting an interspinous process spacer for maintaining separation between adjacent superior and inferior spinous processes of two adjacent vertebrae, the method comprising:
introducing an expandable member between the adjacent superior and inferior spinous processes;
introducing the expandable member percutaneously or arthroscopically while the expandable member is in a unexpanded configuration;
expanding the expandable member to a geometry corresponding to a desired space to be occupied between the adjacent superior and inferior spinous processes;
introducing a measured amount of a flowable material via a catheter to fill the expandable member to the geometry;
allowing time for the delivered flowable material to cure; and
separating the catheter from the expandable member portion containing the cured material.

2. The method of claim 1 wherein the expandable member is a balloon.

3. The method of claim 1 wherein percutaneous introduction of the expandable member is achieved through an access path of 10 mm or less.

4. The method of claim 1 wherein percutaneous introduction of the expandable member is achieved through an access path of 3 mm or less.

5. The method of claim 1 further comprising:
verifying the orientation and position of the expandable member radiographically or endoscopically.

6. The method of claim 1 wherein the flowable material is a polymer consisting of bone cement, polyurethane, silicon, copolymers of silicone and polyurethane, polyolefins, neoprene, nitrile or combinations thereof.

7. The method of claim 1 further comprising using at least one connecting member for fixation of the interspinous process spacer in the desired space.

9. A method of sizing an interspinous process spacer for maintaining separation between adjacent superior and inferior spinous processes of two adjacent vertebrae, the method comprising:
introducing an expandable sizing member between the adjacent superior and inferior spinous processes; and
introducing a fluid into the sizing member in an amount corresponding to a desired space to be occupied between the adjacent superior and inferior spinous processes, wherein the amount of the fluid is used to determine an amount of flowable material necessary to fill the expandable member to the desired space.

10. The method of claim 9 further comprising:
removing the fluid from the expanded sizing member in order to unexpand the sizing member to facilitate removal; and
removing the sizing member from the desired space.

11. The method of claim 9 wherein the method further comprises measuring or verifying the degree of distraction radiographically or endoscopically prior to unexpanding the sizing member.
